(19) United States
(12) Patent Application Publication
(10) Pub. No.: US 2007/0153969 A1
(43) Pub. Date: Jul. 5, 2007

(54) RADIOTHERAPEUTIC DEVICE

(76) Inventor: Michael Maschke, Lonnerstadt (DE)

Correspondence Address: SCHIFF HARDIN, LLP;PATENT DEPARTMENT, 6600 SEARS TOWER, CHICAGO, IL, 60606-6473, US

(21) Appl. No.: 11/609,415

(22) Filed: Dec. 12, 2006

(30) Foreign Application Priority Data
Dec. 12, 2005 (DE) 10 2005 059 210.4

(51) Int. Cl. A61N 5/00; H05G 1/60

(52) U.S. Cl. 378/4; 600/1

(57) ABSTRACT

A radiotherapeutic device has a radiotherapeutic irradiation unit with a radiation source for generation of radiotherapeutic radiation and a beam guidance and/or beam shaping device in order to direct the radiotherapeutic radiation in a defined manner onto a specific irradiation region. The radiotherapeutic device additionally has an imaging unit that includes a radionuclide emission tomography acquisition unit and a computed tomography scanner. The radiotherapeutic device also has a support device with a positioning device in order to position the support device in an image acquisition position in which a body region to be irradiated of a patient borne on or in the support device is located in an acquisition region of the image acquisition unit, or in order to position the support device in an irradiation position in which the body region to be irradiated of the patient is at least partially located in congruence with the irradiation region of the irradiation unit. The radiotherapeutic device additionally has a coordinate registration device that registers changes of all position coordinates of the support device given a movement of the support device between the image acquisition position and the irradiation position.

### BACKGROUND OF THE INVENTION

1. Field of the Invention

The present invention concerns a radiotherapeutic device of the type having a radiotherapeutic irradiation unit with a radiation source (for example a linear accelerator) for generation of radiotherapeutic radiation, and a beam guidance and/or beam shaping device in order to direct the radiotherapeutic radiation in a defined manner onto a determined irradiation region.

2. Description of the Prior Art

Worldwide, cancer is the second most common cause of death in the significantly developed countries with a rising tendency in other countries (in particular in Asia). Irradiation therapy of tumors and metastases has been established in cancer therapy for years. In the early years radiation treatments were for the most part implemented using radioactive sources. For some years linear accelerators have been used for this that operate utilizing bremsstrahlung and fast electrons. In addition, there are high voltage radiotherapy apparatuses for treatment of less dangerous cancer types. In each radiation treatment it is extraordinarily important for the planning and monitoring of the therapy to have precise information about the size and the location of the tumor and the metastases to be treated as well as about the surrounding tissue and organs. Only in this manner can the tumor be destroyed with a sufficiently high radiation dose and damage to healthy tissue and organs thereby be avoided.

Before such an irradiation treatment, images of the body region of the patient to be irradiated are produced by means of suitable imaging methods (normally by computed tomography), from which images the necessary data for the planning the irradiation treatment can then be extracted, Upon the repositioning of the patient from the imaging system to the radiotherapeutic apparatus, care must therefore be taken that the patient is positioned in a matching manner at the radiotherapeutic apparatus. Only in this manner can the treatment region within the coordinate system of the radiotherapeutic apparatus be exactly established using the position data generated in the coordinate system of the imaging system. The position data specify, for example, the exact point a tumor is located and the dimensions thereof. For this purpose relatively complicated methods are necessary that include complicated markings at or on the patient. The entire method is not only time- consuming but also is very uncomfortable for the patient.

To address this problem, in JP 2001299943 A it is proposed to position a computed tomography apparatus and a radiotherapeutic irradiation device relative to one another such that the patient can be moved the same patient bed both through the computed tomography scanner (in order to generate the necessary exposures) and can be directly positioned in the radiotherapeutic device. A repositioning of the patient is then no longer necessary. A problem is that, although computed tomography scans generate very good anatomical images, they are not always good for use for exact identification of various tumors and metastases.

A better diagnosis possibility for identification of tumors and metastases is achieved with positron emission tomography). PET methods have already been established for years in nuclear medicine. Small quantities of specific substances provided with radioactive materials (known as tracers) are injected into the human body in order to detect various metabolisms in the body by measurement of the radioactive radiation. The quantity of the injected substance is extremely low and lies in the sub-physiological range. It therefore does not influence the metabolic processes to be examined and also does not lead to toxic reactions. The weakly radioactive radiation (y-radiation) is registered by scintillator detectors and from this an image is generated. The tracer accumulates in specific organs and/or tumors and thus allows a very good diagnosis of the metabolisms and in particular a very easy and exact detection of tumors and metastases in surrounding tissue. An assessment of the blood circulation of, for example, the heart muscle is also possible with PET. The radiation emitted by the tracer in the tumor is isotropic, meaning that the y-radiation is uniformly emitted in all directions. Preferred radionuclides with a short half-life are used for PET. One example is O₁₅, which exhibits a half-life of 2 minutes. A further frequently used tracer is 18-FDG (fluordeoxyglucose)

An imaging technology similar to this, which likewise operates with radionuclides, is SPECT (single photon emission computed tomography), but this method has only been known for a few years. The radionuclides used for this likewise emit individual y-quanta upon decay. However, relative to the radionuclides used in the PET method these radionuclides have the advantage that they exhibit a longer half-life and therefore do not have to be introduced in immediate proximity to the examination location. Typical tracers for use in SPECT acquisitions are Tc99m-MDP (Tc=technetium) for bones as well as TI 201 or Tc99M-MIBI for the examination of heart blood circulations or iodine 131 for tumor detection.

An association of the acquired image data in the coordinate system of the radiological irradiation device is required in order to be able to use such very precise tumor detection methods for the planning of tumor irradiation treatments. Insofar as (as described above) a device is used in which a computed tomography scanner is coupled with the radiotherapeutic system, software-based registration can be used for this, whereby the images generated by means of the radionuclide emission tomography method are superimposed on the computed tomography images. Various methods for such a software-based registration of PET images and CT images are known to those skilled in the art. A significant disadvantage of a software-based registration, however is that conventionally it can be implemented only by means of manual interactions of an expert, which requires corresponding personnel and time. Further uncertainties thereby result, for example the possibility of the patient moving slightly and thus altering his or her position during the long time span between the exposures and the actual treatment.

### SUMMARY OF THE INVENTION

An object of the present invention is to provide a radiotherapeutic device of the aforementioned type in which the position and the dimensions of tumors or other subjects to be irradiated can be detected in an optimally fast manner with optimally high safety and the irradiation can be implemented very exactly corresponding to this information.

This object is achieved in accordance with the invention by a radiotherapeutic device having a radiotherapeutic irradiation unit and an imaging unit that includes a radionuclide emission tomography acquisition unit and a computed tomography data acquisition unit Moreover, the radiotherapeutic device has a patient support device (for example a patient bed, a seat or the like) with a positioning device fashioned such that the support device can be positioned either in an image acquisition position (in which a body region to be irradiated of a patient borne on or in the support device is located in an acquisition region of the imaging unit) or in an irradiation position in which the body region to be irradiated of the patient is located at least partially in congruence with the irradiation region of the radiotherapeutic radiation unit, The radiotherapeutic device also has a coordinate registration device in order to register the change of all position coordinates of the support device given a movement of the support device between the image acquisition position and the irradiation position

A significant advantage of the inventive radiotherapeutic device is that it allows a production of exposures of the appertaining body region of the patient by means of a method with which precisely those subjects with which the subsequent irradiation treatment deals can be detected and dimensioned with optimally high precision. The data thereby acquired thus can thereby be (immediately) used in the irradiation without human interactions (which naturally can have errors and time losses associated therewith) being necessary.

In a preferred embodiment the radionuclide emission tomography acquisition unit is a PET acquisition unit. As already described, PET acquisition methods have been established for a long time in medical imaging and thus a rich wealth of experience in the application of such imaging methods exists.

In a further preferred embodiment, the radionuclide emission tomography acquisition unit is a SPECT acquisition unit. The advantage of a SPECT method is that the tracers do not have to be immediately generated on site since they have a significantly longer half-life.

In another preferred embodiment, the detector unit of the radionuclide emission tomography acquisition unit is fashioned such that it can be used both for measurements in SPECT acquisition methods and for measurements in PET acquisition methods In principle the same scintillator detectors can be used in both methods. For use in a SPECT imaging method the scintillator detectors must only be additionally equipped with a collimator in order to acquire the directional information.

As long as, in accordance with the invention, all position coordinates are registered given movement or repositioning of the support device between the image acquisition position and the irradiation position, it is ultimately insignificant which path the support device must cover between the image acquisition position and the irradiation position. It is likewise also insignificant whether the movement of the support device ensues manually or automatically. In the manual case the coordinate changes can be detected, for example, by suitable sensors. Given an automatic control the necessary coordinate changes already exist in the controller and can simply be adopted.

However, the radiotherapeutic device with its radiotherapeutic radiation unit and its imaging unit is advantageously designed such that the irradiation region and the image acquisition region are arranged at varying positions along a measured axis and the support device is borne such that it can move linearly along this axis. In this case only the change of the position coordinate of the bearing device along the appertaining axis must be registered.

For this purpose the radiation source of the radiotherapeutic irradiation unit is advantageously borne such that it can rotate around a first isocenter. This isocenter lies in the irradiation region, or ultimately forms the irradiation region. The radionuclide emission tomography acquisition unit is likewise equipped with a detector unit that is arranged annularly around a second isocenter or has at least one detector element rotating around a second isocenter. The first isocenter and the second isocenter are thereby arranged on the common axis along which the support can be moved.

In another preferred embodiment the imaging unit has a computed tomography data acquisition unit in addition to the radionuclide emission tomography acquisition unit. The radionuclide emission tomography acquisition unit and the computer tomography acquisition unit are thereby particularly preferably located in a common housing. However, either the acquisition regions of the two acquisition units should at least lie in congruence or be arranged next to one another (achievable by the patient positioning device). This preferred embodiment combines the advantages of the various imaging methods. In principle better anatomical images of the patient can thus be achieved with a computed tomography method than with a radionuclide emission tomography method which (as explained above) offers better results in the detection and dimensioning of tumors. Both computed tomography images and, for example, PET or SPECT images can be generated with a radiotherapeutic device so equipped, which unifies in the imaging unit a radionuclide emission tomography acquisition unit and a computed tomography acquisition unit. The images can then be superimposed on one another by means of a hardware-based registration method that can be implemented wholly automatically in order to achieve ideal images for the further planning of the irradiation. Combined PET/CT apparatuses are known, for example, from DE 103 39 493. In a similar manner as described therein a combination of a computed tomography apparatus with a radionuclide emission tomography acquisition unit can also ensue in the inventive radiotherapeutic device.

The radionuclide emission tomography acquisition unit and the computed tomography acquisition unit preferably have a common detector unit. Significant cost savings are thereby possible.

The radiotherapeutic device, moreover, can include an image fusion unit in order to combine images acquired by means of the radionuclide emission tomography acquisition unit with computed tomography images (advantageously with the images acquired by means of its own computed tomography acquisition unit) or with magnetic resonance images into overall images, i.e. in order to suitably superimpose the images.

The radiotherapeutic irradiation unit and the imaging acquisition unit of the radiotherapeutic device preferably operate in a common coordinate system. To the extent it is more advantageous (in terms of control technology and/or a cost point of view) for the two sub-units to use separate coordinate systems, the radiotherapeutic device has a suitable coordinate processing device in order to automatically convert position coordinates of the body region of the patient to be irradiated between the coordinate systems on the basis of the registered position coordinate changes of the patient positioning device.

The coordinate registration device can include a movement sensor system for detection of a movement of the patient positioning device. Manual movements of the support device can also be safely registered with this. Such a movement sensor system has advantages even given an automatic control of the bearing device, since monitoring of the data output by the automatic control device is possible in order to reliably exclude errors that could occur due to incorrect coordinate detection or transfer.

As mentioned above, the support device can be automatically actuated. The positioning device preferably has a controller in order to activate the actuator coordinated with the irradiation unit. This means that the actuator and the positioning device of the support device are used not only for movement of the patient from the acquisition region into the irradiation region but also are used in order to move the irradiation region (which is typically a very small punctiform region) within the radiotherapeutic irradiation unit relative to the unit, or conversely, to move the patient relative to the irradiation region such that ultimately the entire region to be irradiated (for example a tumor with a very complex shape) is exactly irradiated and destroyed by the shifting of the irradiation region without severely damaging the surrounding tissue.

The inventive device can additionally include an ablation unit (for example a catheter) with which tumors, metastases and other subjects to be removed can be locally destroyed (for example by overheating with radio-frequency radiation or lasers, or by supercooling by means of cryo-methods, or by the introduction of drugs. A combined treatment of the patient with high-engery radiation and with a classical ablation method is then possible.

The radiotherapeutic device also can include an ultrasound imaging unit. This ultrasound imaging unit enables the generation of further images usable for the radiotherapeutic treatment. It also enables monitoring of the positioning of a catheter of an ablation unit.

The radiotherapeutic device can also include a movement sensor system for detection of patient movement signals that represent a movement of the patient or a movement of body parts of the patient relative to the patient positioning device. The patient movement signals can then be used for correction of the position data determined from the imaging unit

As before, a problem is irradiation of tumors that are located within or in proximity to moving organs. This concerns all tumors in the chest and abdominal cavity of the patient since the heart movement and the breathing movement continuously alter the entire organs and thus also the position of the tumors. The radiotherapeutic device therefore preferably has an organ movement sensor system in order to detect organ movement signals that represent a movement of organs of the patient. Such organ movement sensor systems can be, for example, EKG apparatuses, respiration sensors etc. with which corresponding organ movement signals can be determined. Such sensor systems are known to those skilled in the art (in particular from critical care medicine) for monitoring the vital functions of a patient.

The radiotherapeutic device can include a synchronization unit that activates the imaging unit on the basis of the organ movement signals such that exposures of the body region of the patient to be irradiated are generated in a specific movement state. The synchronization unit then activates the radiotherapeutic irradiation unit on the basis of the organ movement signals such that the body region of the patient to be irradiated is irradiated in a specific movement state. An example of this is to monitor the heart and respiration movement of the patient with an ECG unit in the data acquisition and the signals are used for gating in order to generate exposures of the tumor in a specific movement state of the heart and the lung. The patient is then subsequently moved from the imaging unit to the radiotherapeutic irradiation unit, and as before ECG signals are acquired In the irradiation a corresponding gating then ensues via the synchronization unit with the use of these ECG signals, such that the irradiation at the images acquired from the imaging unit, or at the coordinates calculated therefrom, always ensues when the organs are in the same movement state as in the image generation.

### DESCRIPTION OF THE PREFERRED EMBODIMENTS

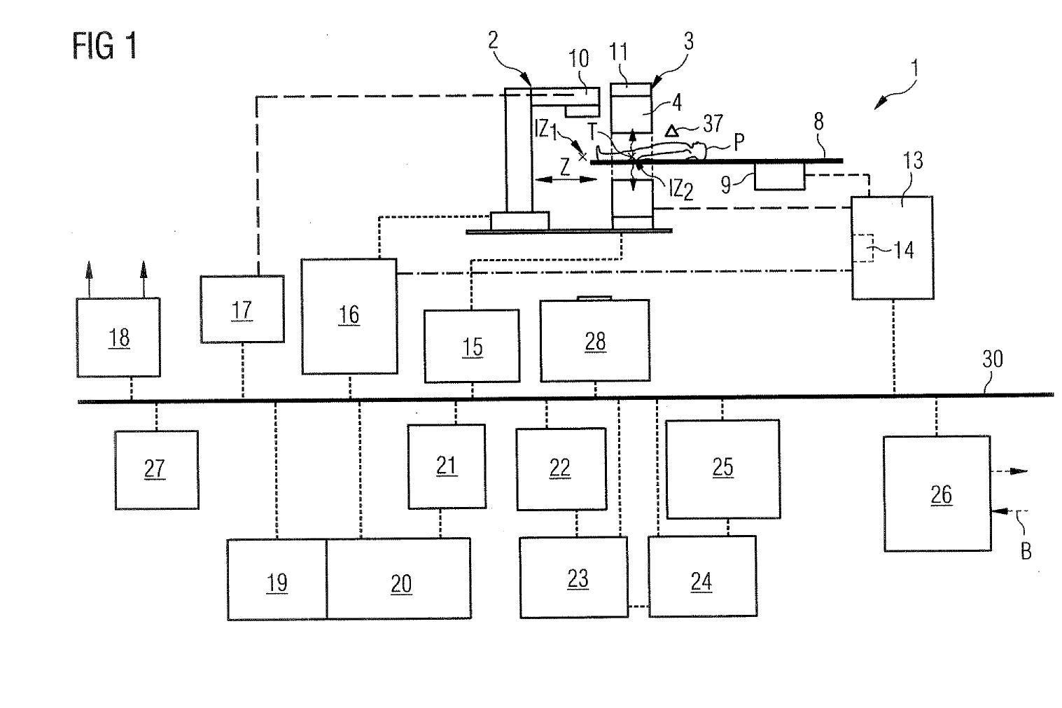

FIG. 1 is a block diagram of a first exemplary embodiment of an inventive radiotherapeutic device, including peripheral apparatuses.

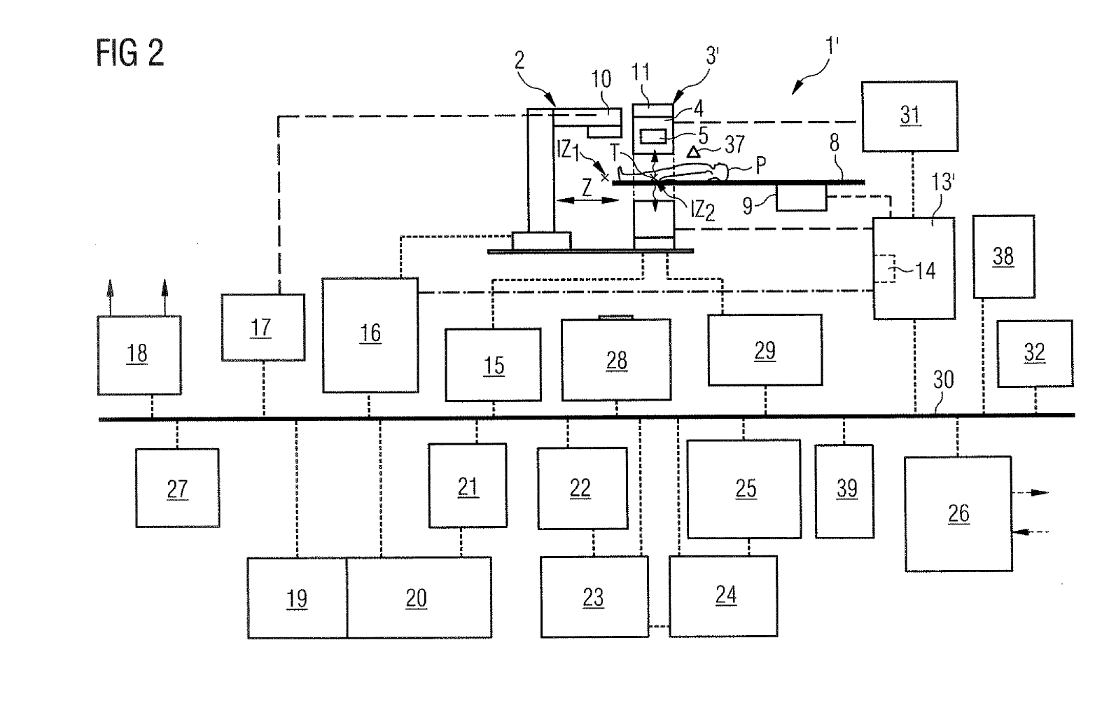

FIG. 2 is a block diagram of a second exemplary embodiment of an inventive radiotherapeutic device including peripheral apparatuses.

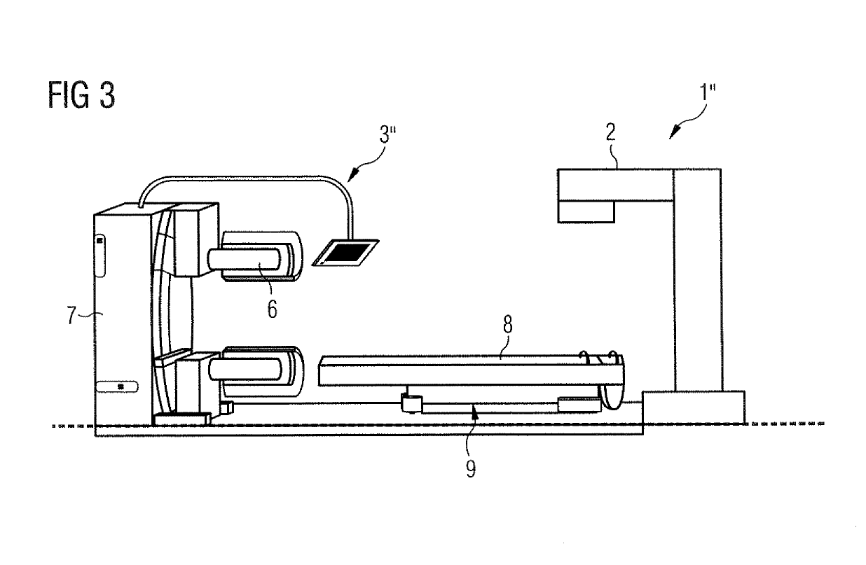

FIG. 3 shows a third exemplary embodiment of an inventive radiotherapeutic device.

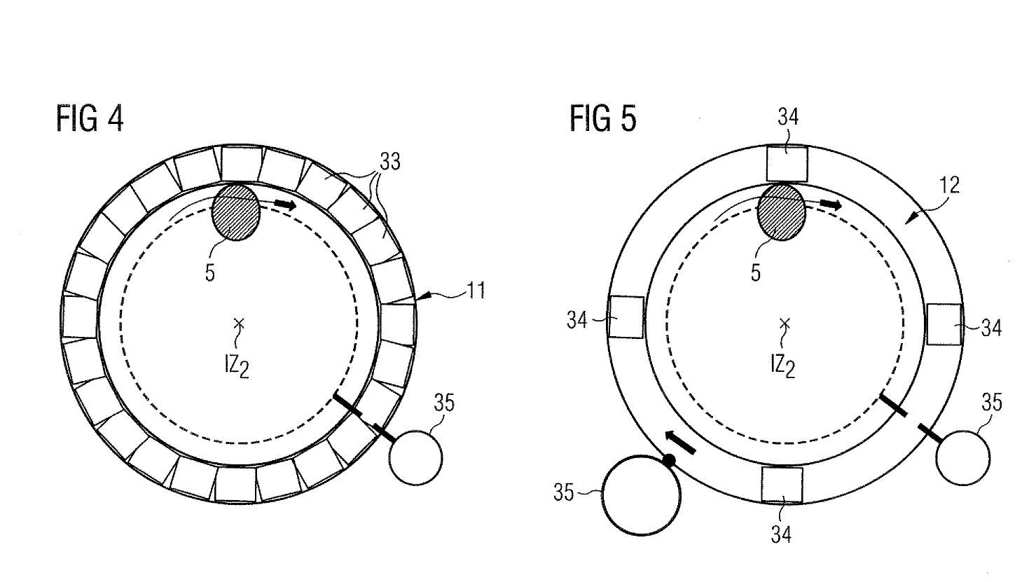

FIG. 4 schematically illustrates the functional basis of a combined CT/PET acquisition unit according to a first exemplary embodiment.

FIG. 5 shows the functional basis of a combined CT/PET acquisition unit according to a second exemplary embodiment.

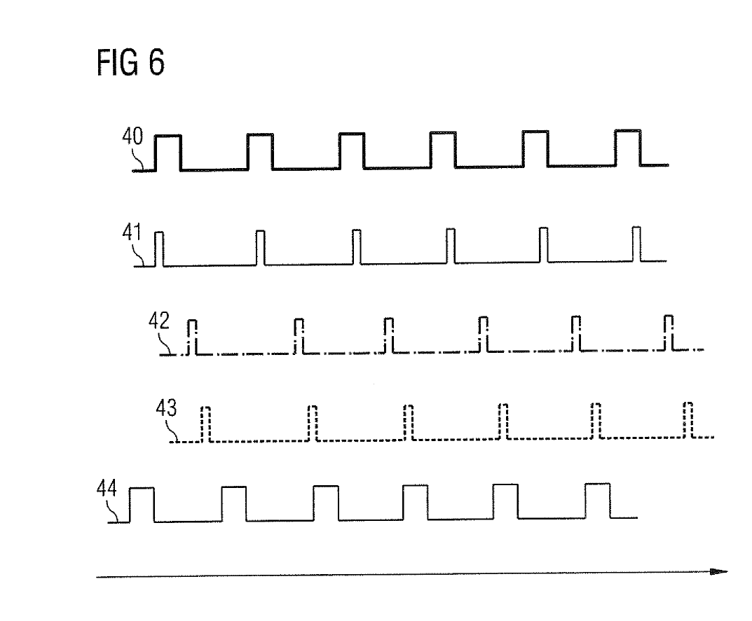

FIG. 6 shows the temporal relation of the detector readout times and irradiation pulse times in a preferred synchronization of the various actions of the inventive device.

### DESCRIPTION OF THE PREFERRED EMBODIMENTS

In the exemplary embodiment shown in FIG. 1 the radiotherapeutic device 1 has a radiotherapeutic irradiation unit 2 with a linear accelerator 10 for generation of a high-energy electron or ion beam. This high-energy beam is appropriately shaped and directed by suitable beam guidance or beam shaping devices, for example lamella diaphragms or the like Suitable techniques and devices for this are known to those skilled in the art and therefore need not but shown. The irradiation unit 2 is designed so that the radiation source rotates around an isocenter IZ₁, (which corresponds to the irradiation region) and thus the beam strikes on the irradiation region from various directions in temporal succession. In this manner it is ensured that a very high intensity is achieved in the irradiation region in the isocenter IZ₁, while the intensity is significantly lower in the surrounding tissue.

A patient P can be moved relative to the isocenter IZ₁, of the irradiation unit 2 by means of a patient bed 8. The irradiation region (which is a relatively small point in space) thus can be moved bit by bit over time through the entire region to be irradiated in order, for example, to destroy a tumor in the body of the patient P as much as possible.

An imaging unit 3 is arranged parallel to the irradiation unit 2. In the shown embodiment the imaging unit 3 is an individual PET acquisition unit 4. Alternatively, a different radionuclide emission tomography acquisition unit (for example a SPECT acquisition unit) can be used. The PET acquisition unit 4 has a detector ring 11 arranged around a second isocenter IZ_{2.}

For acquisition of PET images the patient P is positioned with the patient bed 8 and the positioning device 9 such that the isocenter IZ₂ (i.e. the acquisition region of the PET acquisition unit 4) is congruent with the region of the patient P to be examined. Moreover, a tracer T (for example O₁₅ or 18-FDG FDG) is injected into the patient P in advance of imaging. The tracer T strongly accumulates in the organs or in the tumor tissue of interest. The radionuclides within the tracer T decay over time and thereby emit y-rays. In each decay event exactly two y-quanta are emitted in exactly opposite directions, the quanta being detected by the detector ring 11. This means that events occurring on opposite detector sides are measured in coincidence. Direction information (i.e. the direction the appertaining y-quanta have struck the detector) is determined based on this coincidence and from this information the location of the decay can be reverse calculated. An image in which tumors, metastases etc. can be detected particularly well is generated in this manner with the typical methods.

The treatment of a patient P in such a radiotherapeutic device 1 can ensue as follows:

The patient P is initially positioned on the patient bed 8 and moved into the PET acquisition unit 4 for imaging. There the PET exposures are generated. The region to be irradiated within the body of the patient P is then exactly established on the basis of these exposures With the positioning device 9 the patient bed 8 is subsequently moved along a z-axis on which the isocenters IZ₁, IZ₂ of the PET acquisition unit 4 and the radiotherapeutic irradiation unit 2 lie and the patient P is thus positioned in the region of the irradiation unit 2. An activation of the positioning device 9 thus ensues to cause the region defined with the aid of the PET exposure, in which region the tumor is located to be irradiated. The movement of the patient bed 8 is monitored with a movement detector.

A further movement sensor 37, which detects the movements of the patient P on the patient bed 8, is located above the patient bed 8. Such a movement sensor 37 can be based on various operating principles. For example, such a movement sensor 37 can operate in an electrical, capacitive, magnetic, acoustic or visual manner. An alternative is also a “mathematical movement detector” which, for example, detects a movement of the patient P from image signals of the imaging unit 3. These movement data can then be utilized in order to implement corrections in the determination of the position of tumors on the basis of the generated PET exposures.

Control of the entire device 1 requires a number of components. As shown in FIG. 1, the linear accelerator 10 is connected to a linear accelerator controller 17. The PET acquisition unit 4 likewise has a system control device 13. The movement of the irradiation unit is moreover monitored by a movement control unit 16. This is in turn connected with the system control device 13 for the PET acquisition unit 4 and thus with the positioning device 9 and the patient bed 8. A coordinate registration device 14 is also located within the system control device 13 for the PET acquisition unit 4 in order to precisely register the change of the position of the patient bed 8 when the patient bed 8 is moved between the image acquisition position within the PET acquisition unit 4 and the irradiation position at the irradiation unit 2. The coordinate registration device 14 can be, for example, in the form of suitable software within a processor of the system control device 13.

The PET acquisition unit 4 is moreover connected with PET data pre-processing unit 15 in which the PET image raw data are prepared for the further evaluation.

During the image acquisition or the subsequent irradiation, the patient P is monitored by physiological sensors, (for example an ECG apparatus, a pulse sensor, a respiration measurement apparatus, a blood pressure apparatus etc.), (not shown) which are connected to a physiology signal processor 28. In order to avoid breathing artifacts, for example, a chest belt can be used that determines the breathing amplitude and frequency. The patient's pulse can be determined by evaluation of the ECG signal or of the blood pressure curves.

All previously cited components are connected among one another as well as with further components of the radiotherapeutic device 1 via a bus system 30. Among these further compounds are, among other things, an image processing unit 22 that reconstructs the PET exposures, and an operator interface 22 (for example a typical console or a terminal) for operation of all components of the radiotherapeutic device 1. A display unit 19 that displays the acquired images or further information to the operator is also coupled with this operator interface 20.

The radiotherapeutic device has a treatment planning unit 21 as a further component. This can also be part of the operator interface 20 and serves to plan the treatment with the aid of the operator interface 20 and on the basis of the generated PET images and to specify specific regions onto which the radiotherapeutic radiation should be directed in the radiation treatment.

The radiotherapeutic device 1 also has a movement and gating controller 18 that receives the data of the movement detector 37 as well as the data of the physiology signal processing 28. With this movement and gating controller 18 (serving as a synchronization device 18) it can be ensured that the irradiation of the patient ensues to the greatest extent possible in the same movement states of the individual organs in which the PET exposures were generated, in order to thus ensure with the greatest possible assurance that the irradiation region is also located in the tumor tissue and not in the adjoining, surrounding tissue.

Moreover, an image and data archive 27 and a DICOM interface (DICOM=digital imaging and communication in medicine) are connected to the bus system 30 in order to exchange the patient data and image data with other systems, for example via a radiological information system RIS or an image data archiving and communication system, such as a PACS (PACS=picture archiving and communication system).

Moreover, computed tomography exposures or magnetic resonance images B of the patient P that were previously produced before the radiation treatment can also be transferred via the DICOM interface 26. These can then be superimposed on the PET exposures by an image fusion device 25. This ensues utilizing a calibration unit 23 and an image correction unit 24 that calibrates the images with respect to one another and effects necessary corrections in order to thus generate overall images that can be optimally utilized for the treatment planning, both with regard to anatomical diagnosis and with regard to the tumor determination. The necessary interactions of the operator for such a software-based registration ensue via the user interface 20.

At this point it is noted that the various components (in particular the PET acquisition unit 4 and the irradiation unit 2) naturally also include all further sub-components that are typically necessary for the operation of such apparatuses, such as, for example, one or more power supply units that serve for energy supply of the various shown components. For reasons of better clarity these are not shown in detail in the figures.

FIG. 2 shows a variant of the radiotherapeutic irradiation device 1 according to FIG. 1. This radiotherapeutic device 1′ coincides in large parts with the device 1 previously described. The components identical in both devices are therefore not explained again.

A significant difference in the embodiment of FIG. 2 is that the imaging unit 3′ thereof includes a CT acquisition unit in addition to the PET acquisition unit 4′. This means that the imaging unit 3′ is a combined PET/CT apparatus. An x-ray radiator 5, which rotates around the isocenter IZ₂ is arranged in the image acquisition unit 3, such as in a gantry housing annularly that surrounds the isocenter IZ₂. This is also schematically shown in FIG. 4. The x-ray radiator 5 is moved by a motor 35. The detector units 33 of the detector ring 11 for detection of the PET radiation are designed to also measure the x-ray radiation emitted by the x-ray radiator 5. This means that both PET images and x-ray CT images can be acquired in the same detector arrangement 11. For this purpose the detector units 33 have typical scintillator elements with detector elements and pre-intensifiers arranged behind these scintillator elements in the radiation direction. The design of such detector units is known to those skilled in the art and therefore need not be shown in detail herein. Alternatively it is also possible to use adjacent, separate detector systems.

For operation of the x-ray source 5 the device 1′ has a high-voltage generator 31. The system control device 13′ for the imaging unit 3′ thus is also equipped for control of the high-voltage generator 31 and includes the necessary hardware/software in order to control such a combined PET/CT apparatus. Here as well the system control device 13′ has a coordinate registration device 14.

Moreover, a further CT data pre-processing unit 29 for pre-processing of the CT raw data and an image processing unit 39 in order to reconstruct the CT exposures are provided.

In the radiotherapeutic irradiation device 1′ the CT images and PET images generated with the image acquisition unit 3′ can be immediately (directly) combined in the image fusion device 25, meaning that it is not necessary to draw (via the DICOM interface) upon external, previously produced CT or MR images and adapt these to one another in a manually- supported, software-based registration. If desired, however, via the DICOM interface 26 arbitrary prior CT, MR, PE, SPECT exposures of the patient from preceding examinations can be accessed in order to monitor a tumor growth, for example via a comparison with current exposures.

The device 1′ also includes a tumor ablation unit 32 and a ultrasound imaging unit 38. The ablation unit 32 has a catheter with which radio-frequency or laser radiation can be directed in a targeted manner to the location of the tumor to necrotize a tumor tissue by overheating. Alternatively or additionally, the ablation unit 32 can be equipped with a catheter in order to necrotize the tumor tissue by supercooling with extreme cold (for example liquid nitrogen), or comprise a catheter in order to necrotize the tumor tissue in a targeted manner by injection of drugs.

Further images of the inside of the patient can be generated with the ultrasound imaging unit 38, which has a typical ultrasound head as well as other necessary components. Monitoring of the catheter of the ablation device 32 can ensue with this ultrasound imaging unit 38.

FIG. 3 shows a further combination of a radiotherapeutic irradiation unit 2 with an imaging unit 3″, wherein the imaging unit 3″ has a combinated computed tomography scanner 7 and a SPECT acquisition unit 6. The SPECT acquisition unit 6 and the CT scanner 7 are arranged in parallel in the same housing. With the positioning device 9, the patient bed 8 can be selectively moved into the acquisition region of the CT scanner 7 or into the acquisition region of the SPECT acquisition unit 6.

In another exemplary embodiment the SPECT acquisition unit and the CT scanner use the same detector arrangement. This is schematically shown in FIG. 5. The detector arrangement 12 here has four detector units 34 that are able to measure (detect) both y-quanta and x-ray quanta. With a motor 36 this detector arrangement 12 rotates around an isocenter IZ₂. The a further motor 35 rotates an x-ray radiator 5 rotates around the isocenter IZ₂ within the gantry housing. To measure the y-quanta from the radionuclides of the tracer, the detector units 34 each have a collimator (not shown) that ensures that only the y-quanta are detected that proceed vertically through the collimator and strike the detector. Information about the incoming directions of the respective particles thus can be acquired. A corresponding image thus can be generated in a typical manner by back-projection. The collimators for the SPECT acquisition do in fact reduce the sensitivity of the detectors and thus the image resolution. This is, however, compensated by using tracers with a longer half-life. The collimators can be removed or opened wide to measure the CT exposures.

In principle only one detector element 34 which rotates around the isocenter IZ₂ can be employed in this embodiment, instead of multiple detector elements 34. The use of oppositely-situated detector elements, however, has the advantage that in principle even a measurement of PET exposures is possible with this method, since events producing radiation in coincident directions can be measured.

A typical examination and treatment workflow in an inventive radiotherapeutic device can proceed as follows:

PET or SPECT exposures are initially generated. Insofar as the imaging unit 3, 3′, 3″ is a unit which additionally has a CT acquisition unit, corresponding CT images can be generated. Alternatively, previously-acquired CT or magnetic resonance images can be accessed. The SPECT or PET exposures can then be superimposed with the generated or accessed CT or magnetic resonance exposures, In the subsequent treatment planning, the subject to be treated is then precisely localized and isolated within the images. This information is supplied to the control device of the radiotherapeutic irradiation unit 2 and the patient P on the patient bed 8 is moved to the radiotherapeutic irradiation unit 2. Insofar as the radiotherapeutic irradiation unit 2 utilizes a different coordinate system than the imaging unit 3, 3′, 3″, the coordinates are automatically converted. The irradiation therapy then begins. Additional treatments (for example brachytherapy) can optionally be implemented. The patient P on the patient bed 8 can subsequently be moved back again to the imaging unit 3, 3′, 3″ in order to acquire new images and thus to monitor or modify the treatment success.

It is also possible to generate the imaging units in parallel with a combined imaging unit which also has a CT unit in addition to a radionuclide emission tomography acquisition unit, but then a synchronized readout of the detectors and a synchronized emission of the x-rays is necessary. At the same time a suitable gating can be ensured with the aid of an ECG signal so that the images are respectively generated only in specific movement states. This is explained in the example of a combined PET/CT acquisition in FIG. 6.

FIG. 6 shows various digital control signals with respective time for the synchronized control of an image generation and irradiation process. For example, a high signal level a first control signal 40 effects the readout of the y-quanta for PET acquisition, A high signal level a second control signal 41 effects the readout of an EKG and/or respiration sensor. Furthermore, a third control signal 42 is shown which, at a high signal level, initiates the readout of an x-ray pulse for the CT acquisition. Via a high signal level a fourth control signal 43 here effects the readout of the detectors for detection of the x-ray radiation for the CT acquisition. Finally, FIG. 6 shows a fifth control signal 44 that initiates a radiation pulse of the therapy radiation given a high signal level. With a clocked control designed in such a manner the various signals no not mutually, disadvantageously influence one another.

A synchronization of the therapy radiation with the readout of the PET detectors or of the x-ray detectors of the CT is necessary when, for example, one body region of the patient has already been irradiated in parallel and exposures are still to be generated in another body region. Even when such a parallel acquisition and irradiation does not ensue, a synchronization of the irradiation pulses to an ECG signal is reasonable in order to ensure that the irradiation also ensues in the same movement states as they existed in the production of the exposures. In this case it would be ensured that the irradiation pulse lies (relative to an ECG trigger pulse) at the same position as the readout times for the PET and the CT acquisition.

As the preceding exemplary embodiments show, the inventive radiotherapeutic device 1, 1′, 1″ can be used in a universal manner. It is naturally also possible to use only the irradiation part in specific applications or to use only the imaging unit for individual examinations without subsequent irradiation. Nevertheless, such a combined radiotherapeutic device has the advantage that many components of the radiotherapeutic irradiation unit and of the imaging unit can be shared This in particular applies to the user interface. Given use of a combined imaging unit made up of radionuclide emission tomography acquisition unit and CT unit, good anatomical images and functional images can additionally be generated with the same apparatus, so registration problems are avoided.

The designs described in detail in the proceeding as well as the described method workflow are only exemplary embodiments, which can be modified by those skilled in the art without departing from the scope of the invention. In particular the shown systems can include further components and equipment (for example protective walls or protective curtains) in order to prevent scattering of radiation from one component into the other components, or to additionally protect medical personnel or the patient from scatter radiation. The spatial arrangement of the radiotherapeutic irradiation unit (the imaging unit) and the patient bed relative to one another also can be different from that shown in the figures, independent of whether the radiotherapeutic irradiation unit is a SPECT, PET, SPECT/CT, or PET/CT unit. The patient bed thus can be arranged between the irradiation unit and the imaging unit, or laterally next to one of these units. It is only essential that the patient can be correctly positioned by means of the patient bed both in the imaging unit and in the irradiation unit.

Moreover, it is noted that although the invention is described primarily in the field of tumor radiation treatment, its use is not limited to such applications. The invention can likewise be used not only on human patients but also for treatment of animals.

I claim as my invention:

1. A radiotherapeutic device comprising:
a radiotherapeutic irradiation unit comprising a radiation source that emits radiotherapeutic radiation and a beam interaction device, selected from the group consisting of beam guidance devices and beam shaping devices, that directs said radiotherapeutic radiation in a targeted manner onto a predetermined irradiation region;
an imaging unit comprising a radionuclide emission tomography acquisition unit and a computed tomography data acquisition unit;
a support device adapted to receive a radiotherapeutic subject thereon, and a positioning device that interacts with said support device to position said support device in an image acquisition position at which a body region, to be irradiated with said radiotherapeutic radiation, of the subject is located in an acquisition region of the image acquisition unit, and to position the support device in an irradiation position in which said body region is at least partially located in congruence with said irradiation region, said support device successively occupying different position coordinates as said support device is moved by said positioning device; and
a coordinate registration device that registers all changes of said position coordinates of said support device during movement thereof between said image acquisition position and said radiation position and controlling said positioning device to ensure imaging and irradiation of the same body region.

2. A radiotherapeutic device as claimed in claim 1 wherein said radionuclide emission tomography acquisition unit is a PET acquisition unit.

3. A radiotherapeutic device as claimed in claim 1 wherein said radionuclide emission tomography acquisition unit is a SPECT acquisition unit.

4. A radiotherapeutic device as claimed in claim 1 wherein said radiotherapeutic irradiation unit and said imaging unit are disposed relative to each other so that irradiation region and said image acquisition region are located at successive positions along an axis, and wherein said support device is moved by said positioning device linearly along said axis.

5. A radiotherapeutic device as claimed in claim 4 wherein said radiotherapeutic irradiation unit comprises a radiation source mounted to rotate around a first isocenter, and wherein said radionuclide emission tomography acquisition unit comprises a detector unit that is oriented relative to a second isocenter, said first isocenter and said second isocenter being disposed on said axis.

6. A radiotherapeutic device as claimed in claim 4 wherein said detector unit comprises a plurality of detector elements annularly disposed around said second isocenter.

7. A radiotherapeutic device as claimed in clam 5 wherein said detector unit comprises at least one detector element rotatable around said second isocenter.

8. A radiotherapeutic device as claimed in claim 5 wherein said detector unit is a single detector unit shared by said radionuclide emission tomography acquisition unit and said computed tomography data acquisition unit.

9. A radiotherapeutic device as claimed in claim 1 wherein said radionuclide emission tomography acquisition unit produces a first image of said body region and said computed tomography data acquisition unit produces a second image of said body region, and comprising an image fusion unit that combines said first image and said second image to form a combined image of said body region.

10. A radiotherapeutic device as claimed in claim 1 wherein said radiotherapeutic irradiation unit operates according to a first coordinate system and said imaging unit operates according to a second coordinate system, separate from said first coordinate system, and comprising a coordinate processing unit that converts coordinates of said body region between said first and second coordinate system dependent on said position coordinate changes of said support device.

11. A radiotherapeutic device as claimed in claim 1 comprising a movement sensor system that detects said movement of said support device and supplies a signal representing said movement to said positioning device.

12. A radiotherapeutic device as claimed in claim 1 wherein said support device comprises an automatic actuator, and wherein said positioning device comprises a controller that controls said actuator coordinated with activation of said radiotherapeutic radiation by said radiotherapeutic irradiation unit.

13. A radiotherapeutic device as claimed in claim 1 comprising an ablation unit disposed relative to said support device to allow interaction of the ablation unit with the subject on the support device.

14. A radiotherapeutic device as claimed in claim 1 comprising an ultrasound imaging unit disposed relative to said support device to allow interaction of the ablation unit with the subject on the support device.

15. A radiotherapeutic device as claimed in claim 1 comprising a subject movement sensor system that detects physiological movement of the subject relative to said support device, and that generates a physiological movement signal representing said physiological movement, and a control unit supplied with said physiological movement signal and said changes in said position coordinates to control at least one of said radiotherapeutic irradiation unit and said positioning device during irradiation of the subject with said radiotherapeutic radiation.

16. A radiotherapeutic device as claimed in claim 1 comprising a subject movement sensor system that detects organ movement of the subject relative to said support device, and that generates an organ movement signal representing said organ movement, and a control unit supplied with said organ movement signal and said changes in said position coordinates to control at least one of said radiotherapeutic irradiation unit and said positioning device during irradiation of the subject with said radiotherapeutic radiation.

17. A radiotherapeutic device as claimed in claim 17 wherein said control unit controls said imaging unit on the basis of said organ movement signal to acquire said image of the body region at a predetermined movement state, and controls said radiotherapeutic irradiation unit to activate emission of said radiotherapeutic irradiation at the same predetermined movement state at which said image of said body region was obtained.
